(19) United States
(12) Patent Application Publication
(10) Pub. No.: US 2009/0209809 A1
(43) Pub. Date: Aug. 20, 2009

(54) Device for Measuring a Position of a Surgical Instrument

(75) Inventors: Daniel Schäller, Tubingen (DE); Matthias Voigtländer, Gomaringen (DE)

Correspondence Address: DICKSTEIN SHAPIRO LLP, 1825 EYE STREET NW, Washington, DC, 20006-5403, US

(73) Assignee: ERBE Elektromedizin GmbH, Tubungen (DE)

(21) Appl. No.: 11/573,493

(22) PCT Filed: Aug. 5, 2005
(86) PCT No.: PCT/EP05/08531

§ 371 Date: Feb. 23, 2009

(30) Foreign Application Priority Data
Aug. 12, 2004 (DE) 10 2004 039 202.1

(51) Int. Cl. A61B 1/012

(52) U.S. Cl. 600/103

(57) ABSTRACT

When a surgical instrument is inserted into an operating duct of an endoscope, the operator must know when the distal end of the instrument emerges from the distal end of the operating duct and thus reaches the visible zone of the viewing device of the endoscope. The invention provides a device for this purpose that comprises a generator to produce a measurement signal, devices to connect the measurement signal to the operating duct and/or instrument and/or the endoscope, and a measuring apparatus which measures a positional effect that the instrument exerts upon the measurement signal depending upon its position and which generates a display signal dependent upon the effect.

### RELATED U.S. APPLICATIONS

Not applicable.

### STATEMENT REGARDING FEDERALLY SPONSORED RESEARCH OR DEVELOPMENT

Not applicable.

### REFERENCE TO MICROFICHE APPENDIX

Not applicable.

### FIELD OF THE INVENTION

The invention relates to a device for measuring a position of a surgical instrument relative to an operating duct of an endoscope into which the instrument is inserted.

### BACKGROUND OF THE INVENTION

Devices for measuring the position of an operating instrument inside the human body are well known from DE 35 36 271 C1, DE 101 09 310 A1, DE 100 58 370 A1, DE 101 34 911 A1, DE 697 11 311 T2, DE 199 55 346 A1 or DE 697 19 030 T2. All these devices are extremely expensive and are unlikely to be suitable for measuring a position of a surgical instrument relative to an operating duct of an endoscope, into which the operating instrument is inserted.

An endoscope having an inserted instrument is known from DE 198 58 375 A1, which describes an electrode that is movable within the instrument. A sensor is provided for ascertaining the position of the instrument, via which a coagulant can be made to flow, or not to flow. Determination of the position in this case depends upon an appropriate switching contact defined in the instrument.

### BRIEF SUMMARY OF THE INVENTION

The object of the invention is to provide a device according to the above cited prior art wherein the position of the surgical instrument relative to the operating duct of an endoscope, into which the instrument is inserted, is able to be ascertained reliably and in simple manner.

This object is fulfilled by a device which provides a generator to produce a measurement signal, means of interconnecting the measurement signal to the operating duct and/or the instrument and/or the endoscope along with a measurement means for measuring a positional effect that the instrument exerts upon the measurement signal dependent on its position in the operating duct and for generating a display signal dependent upon the effect.

An aim of the invention is thus fulfilled in that an interaction between the instrument and the endoscope and its operating duct is established that is a measure of how far the endoscope is inserted into the operating duct.

The interaction may be an electrical interaction or a mechanical interaction, in particular a pneumatic or acoustic interaction.

In a first preferred embodiment of the invention the generator is configured so that it generates an AC signal or a pulse-shaped DC signal (with high frequency components) as the measurement signal and the measuring apparatus measures the impedance between the instrument and at least a part of the endoscope and/or at least sections of a wall of the operating duct as a positional effect. In order to ensure that the alternating current is not accompanied by any dangers to a patient, the measurement signal must contain at least one frequency of over 300 kHz, since at these frequencies stimulation of the neuro-muscles no longer occurs. Likewise the maximum voltage level must be limited, in order that no thermal damage is provoked. In the case of this embodiment of the invention the instrument in combination with the (electrically conducting) operating duct or to be precise its walls or a separately embedded conductor inside the walls thus forms a capacitance or high frequency transmission line, so that the capacitance or to be precise the length of conductor may be determined by the distributed capacitance along it. An oscillator circuit or a PLL circuit is suitable for determining the capacitance, in which the impedance is arranged to be the variable element for determining a resonant circuit in the oscillator or PLL circuit.

When the surgical instrument comprises a probe having an electrode, it is preferable to measure the impedance between the electrode and the endoscope and/or operating duct wall. No special measurement electrode is required for this.

The measurement arrangement for measuring a static or alternating pressure of a gas in the operating duct and/or in a lumen in the instrument is configured to carry out the pneumatic and acoustic measurement principles. The generator for producing a static or alternating pressure in the operating duct and/or at a distal end of the operating duct and/or in the probe as measurement signal may be constructed easily. A gas flow control device for example is taken as the generator, which conveys gas to the operating duct, so the back pressure and thus the flow resistance in the operating duct depends upon the length over which the instrument is inserted into the operating duct and reduces its cross section by so doing. When the measuring apparatus measures acoustic properties of the system, in this way the radiation impedance of the operating duct (or of a lumen in the instrument) or however a resonant frequency in the operating duct can be measured. Such measurement instruments are easy to construct. The measurement signals are harmless for the patients.

In one embodiment of the invention the measuring apparatus comprises a microphone or similar measurement converter, which is accommodated at the proximal end of a lumen in an instrument. The microphone functions so to speak as a “probe microphone”, which measures the sound introduced into the operating duct.

In the case of a pneumatic measurement the measuring apparatus comprises a pressure sensor, which is accommodated preferably at a proximal end of a lumen in the instrument, the generator being configured to produce a gas pressure in the operating duct and/or in a body cavity, into which the endoscope is to be inserted. It can be determined via this pressure measurement where the instrument is located in the operating duct and, in particular, whether the instrument emerges from the distal end of the operating duct.

The measuring apparatus can be configured to acquire the absolute position of the instrument inside the operating duct. Alternatively the measuring apparatus comprises a change detector for determining a change in the measurement signal during a change of position of the instrument in the operating duct. Then if the instrument emerges again from the operating duct (from its distal end), no further change would be indicated and the user will know where the instrument is located.

According to the invention the above-cited problem is overcome by a method for measuring a position of a surgical instrument relative to an operating duct of an endoscope into which the surgical instrument is inserted, comprising the following steps:

generating a measurement signal;

interconnecting the measurement signal to the operating duct and/or the instrument and/or the endoscope;

measuring a positional effect which the instrument exerts upon the measurement signal according to its position within the operating duct; and

generating a display signal depending upon the effect.

Preferably a device for measuring a position of a surgical instrument relative to an operating duct of an endoscope into which the instrument is inserted, and for generating a display signal depending upon the position, is provided wherein a gas feed, a suction effect, a flow feed or the feeding of a liquid is directed to the instrument or into the operating duct depending upon the display signal. By means of this preferred application various instruments may be operated more reliably than hitherto.

Preferred embodiments of the invention arise from the subordinate claims.

Hereafter embodiments of the invention are illustrated in more detail with the aid of diagrams. Shown here are

### BRIEF DESCRIPTION OF THE DRAWINGS

FIG. 1 a much simplified diagram of an endoscope having an operating instrument partly inserted and peripheral equipment,

FIG. 2 an end section of the endoscope having operating instrument protruding from its distal operating duct end,

FIG. 3 a part circuit of the arrangement according to FIG. 1,

FIG. 4 a further embodiment of the invention having an acoustic measuring apparatus and

FIG. 5 a further embodiment of the invention having a pneumatic measuring apparatus.

### DETAILED DESCRIPTION OF THE INVENTION

In the following description the same reference numbers are employed for the same parts and parts having the same function.

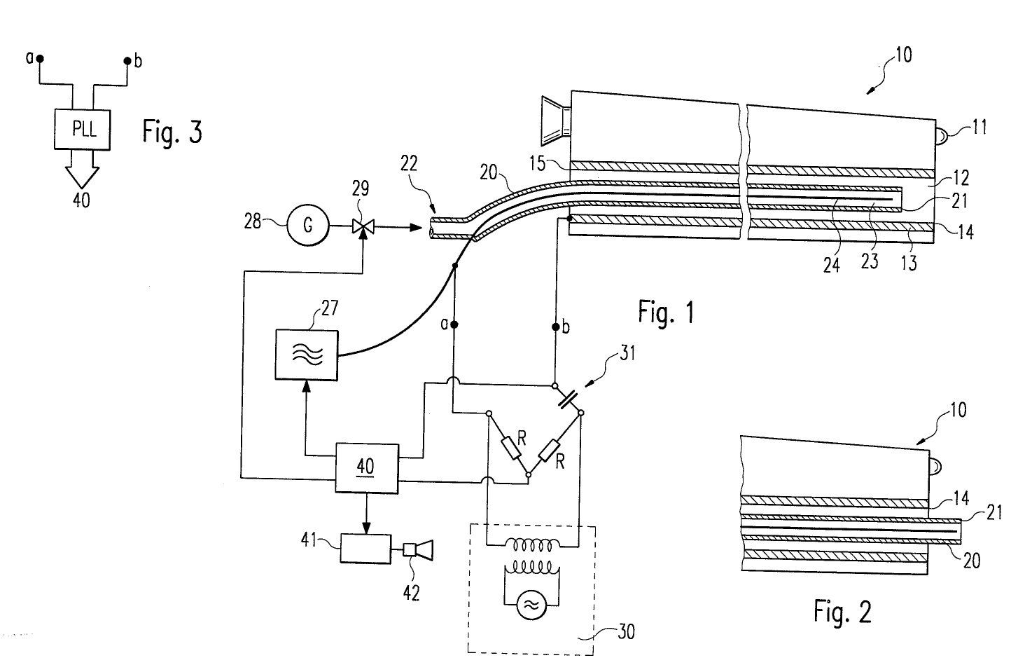

In FIG. 1 an endoscope is shown (much simplified), as it is employed in particular in the medical sector. The endoscope 10 exhibits a lens system 11, which is either connected via a glass fibre cord having an eye-piece arrangement (as shown in the arrangement illustrated) or—and this is in general the case with modern endoscopes—including a CCD camera.

An operating duct 12 is provided in the endoscope 10 (as usual), which exhibits a wall 13. This wall 13 and the endoscope 10 are principally made of metal.

An operating instrument 20 may be inserted into a distal end 14 of the operating duct 12, until a distal end 21 of the operating instrument 20 emerges from the proximal end 15 of the operating duct 12, as shown in FIG. 2. In this position the distal end 21 of the operating instrument 20 is located in the field of view of the lens system 11 of the endoscope 10.

The operating instrument 20 shown here is an APC probe, as is well known from e.g. DE 41 39 029 C2 or U.S. Pat. No. 5,207,675. Such a probe exhibits a lumen 23, via which an inert gas from a gas source 28 may be fed. An electrode 24 is arranged inside the lumen 23, which is connected by its proximal end to an HF surgical appliance. In use this operating instrument 20 must be in a position as shown in FIG. 2, so that the user may bring the distal end 21 of the operating instrument 20 close to a tissue being coagulated. To prepare for this operation it is thus necessary to determine the position of the operating instrument 20 within the operating duct 12 and to adjust it so that the operating instrument 20 and its distal end 21 are located in an operating position and able to be observed by the lens system 11 of the endoscope 10.

In the case of the embodiment of the invention shown in FIG. 1, electrical properties of the system comprising the operating instrument 20 and the endoscope 10 along with its operating duct 12 are employed to generate the required measurement values. Here the electrode 24 of the operating instrument 20 on the one side and the (electrically conducting) wall 13 of the operating duct on the other side are included in a measurement bridge 31, which senses a voltage drop via a capacitor C and two resistors R on the one side and is fed to an evaluation device 40 and a high frequency signal (300 kHz) having defined (low) voltage from a generator 30 is fed into the system via the other side. The capacitor C in this case is preferably chosen so that the bridge 31 is balanced when the operating instrument 20 is inserted fully into the operating duct 12. Instead of such a bridge circuit it is of course possible to use an oscillator circuit, in which the measurement points a and b from FIG. 1 represent the connection points of the frequency determining (capacitive) members and their resonant frequency is measured. A similar circuit is shown in FIG. 3 with a PLL element, whose output signal (which corresponds to the tuning frequency) is fed to the evaluation circuit 40.

The evaluation circuit 40 generates a measurement signal, which corresponds to the distance over which the operating instrument 20 is inserted into the operating duct 12. An indicating device 41 and if necessary a loudspeaker 42 serves as display, in which the arrangement may be made such that an acoustic audible signal is generated, whose pitch corresponds to the insertion depth of the operating instrument 20 in the operating duct 12. Then, when (as shown in FIG. 2) the distal end 21 of the operating instrument 20 begins to protrude from the distal end 14 of the operating duct 12, the capacitance between the electrode 24 and the endoscope 10 and the wall 13 of the operating duct 12 ceases to change, so that the operator can easily observe the emergence of the distal end 21 from the operating duct 12.

It should be stressed at this point that a multitude of electrical measurements is possible to achieve this aim. For example the system can comprise operating instrument 20 and endoscope 10 also understood as a lossy transmission line, whose length is able to be measured with measurement instruments available on the market in known ways per se.

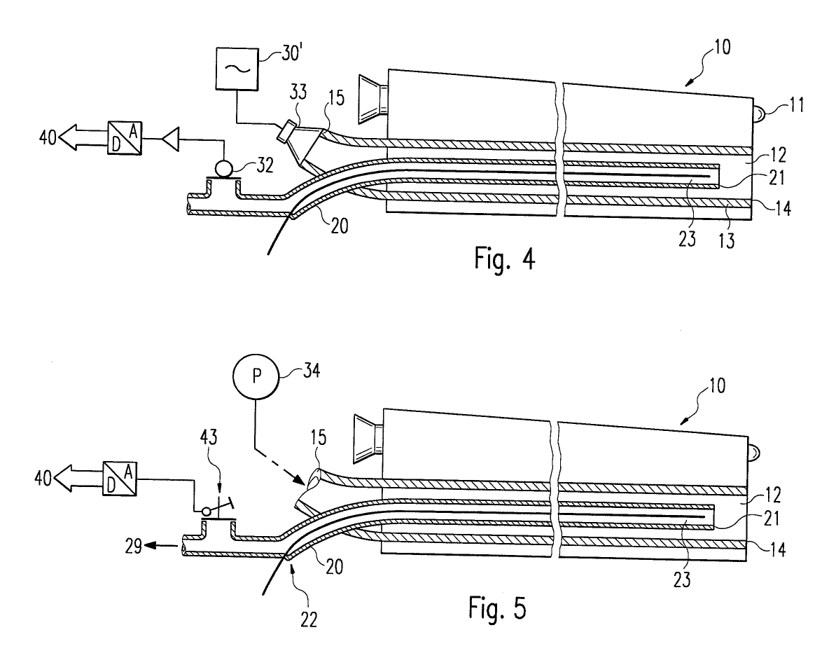

In the case of the embodiment of the invention shown in FIG. 4 an acoustic measurement system is provided. This includes an electro-acoustic converter or loudspeaker 33, which is connected to the proximal end 15 of the operating duct 12 and is fed with an audible signal from a generator 30. The audible signal is detected via an appropriate electromechanical converter, e.g. a microphone 32, with which the lumen 23 of the operating instrument 20 is series connected as for a probe microphone. The output signal of the converter 32 is fed again to the evaluation circuit 40 after appropriate signal conditioning. By means of the sound level it may be ascertained where the distal end 21 is located in the operating duct 12 and in particular whether it protrudes from the distal end 14 of the operating duct 12, since in this range the sound pressure able to be detected sinks abruptly. Naturally it would be possible here to exchange the two acoustic converters 32 and 33, thus feeding the acoustic signal into the lumen 23 of the operating instrument 20 and to measure the acoustic pressure at the distal end 15 of the operating duct 12.

In the case of a further alternative embodiment of the invention not shown here, the acoustic properties of the operating duct 12 with inserted operating instrument 20 are determined. This may for instance be effected by determining the acoustic impedance, which is afforded in the case of the arrangement according to FIG. 4 for the converter 33 and which is dependent upon the penetration depth of the operating instrument 20 in the operating duct 12. Likewise it is possible alternatively to determine an acoustic resonance frequency inside the operating duct 12, which again depends upon the penetration depth of the operating instrument 20. Thus the acoustic measurement is carried out similar to the electrical measurement as described above, while the interaction within the system comprising operating instrument 20 and operating duct 12 is determined.

In the case of the alternative embodiment of the invention shown in FIG. 5, a “static” pressure is determined, which is generated by a pressure source 34, fed into the proximal end 15 of the operating duct 12 and conveyed via the lumen 23 of the operating instrument 20 to a pressure sensor 43 at the proximal end 22 of the operating instrument 20 for measurement. The measurement signal is then again fed to the evaluation device 40. The pressure which appears at the pressure sensor 43 corresponds to the gas pressure at the distal end 21 of the operating instrument 20, while its lumen 23 is shut off by the valve 29 (see FIG. 1), which connects this lumen 23 to the argon gas source 28.

Alternatively it is also possible instead of connecting the gas source 34 to the operating duct 12, to feed gas via a separate duct to a body cavity (e.g. insufflation), into which the endoscope 10 is inserted. The operating instrument 20 would then measure a maximum pressure with the operating duct 12 opened at the proximal end, when the distal end 21 emerges from the distal end 14 of the operating duct 12, since a drop in pressure (due to the through-flow in the operating duct) would no longer occur.

The application of the arrangement shown here or the process shown here is particularly advantageous for automatic control of peripheral equipment of the operating instrument 20, e.g. to control the valve 29, via which inert gas is fed to the lumen 23 in the operating instrument 20 configured as an APC probe. The valve 29 is then released by a separate signal to open, when the distal end 21 of the operating instrument 20 emerges by a sufficiently large amount from the operating duct 12 (see FIG. 2 and the associated description)

The basic principle of the invention arises from the above description, to the effect that the operating duct 12 in the endoscope 10 together with the inserted operating instrument 20 is considered as a total system, so that the interactions between the two parts are able to be utilised in generating a measurement signal.

### LIST OF REFERENCES NUMERALS

10 Endoscope

11 Lens system

12 Operating duct

13 Operating duct wall

14 Distal end

15 Proximal end

20 Operating instrument

21 Distal end

22 Proximal end

23 Lumen

24 Electrode

27 HF-equipment

28 Gas source

29 Valve

30 Generator

31 Measurement bridge

32 Microphone

33 Loudspeaker

34 Pressure source

40 Evaluation device

41 Display (or indicating) device

42 Loudspeaker

43 Pressure sensor

1. Device for measuring a position of a surgical instrument relative to an operating duct of an endoscope into which the instrument is inserted, comprising
a generator adapted to produce a measurement signal;
means connecting the measurement signal to one or more of the operating duct, the instrument and the endoscope;
a measuring apparatus, adapted to measure a positional effect that the instrument has on the measurement signal dependent on its position in the operating duct and adapted to generate a display signal dependent upon said effect.

2. Device according to claim 1, wherein the generator produces an AC signal or a pulse-shaped DC signal as the measurement signal and wherein the measuring apparatus measures the impedance between the instrument and at least parts of the endoscope to determine the positional effect.

3. Device according to claim 2, wherein the measuring apparatus includes an oscillator circuit in which said impedance is a variable element and is adapted to determine a resonant frequency of the oscillator circuit.

4. Device according to claim 2, wherein said instrument comprises a probe with an electrode and said measuring apparatus is adapted to measure the impedance between the electrode and a part of the endoscope.

5. Device according to claim 1, wherein the instrument defines a lumen and the measuring apparatus is adapted to measure a pressure of a gas in at least one of the operating duct and said lumen and wherein the generator is adapted to produce a gas pressure in at least one of the operating duct, a distal end of the operating duct and the lumen as the measurement signal.

6. Device according to claim 5, wherein the measuring apparatus is adapted to measure an acoustic resonance frequency in the operating duct.

7. Device according to claim 5, wherein the measuring apparatus comprises a microphone that is attached to a proximal end of said lumen of the instrument.

8. Device according to claim 5, wherein the measuring apparatus comprises a pressure sensor attached to a proximal end of said lumen of the instrument and wherein the generator is adapted to produce a gas pressure in at least one of the opening duct and a body cavity, in which the endoscope is inserted.

9. Device according to claim 1, wherein the measuring apparatus comprises a detector adapted to determine a change in the position of the instrument in the operating duct.

10. Method of measuring a position of a surgical instrument relative to an operating duct of an endoscope, into which the instrument is inserted, comprising the steps of:
generating a measurement signal;
connecting the measurement signal to one or more of the operating duct, the instrument and the endoscope;
measuring a positional effect that the instrument has on the measurement signal depending upon its position in the operating duct; and
generating a display signal depending upon said effect.

11. Use of a device to measure a position of a surgical instrument relative to an operating duct of an endoscope, into which the instrument is inserted, and to generate a display signal depending upon the position, wherein peripheral equipment is controlled depending upon the display signal.
